(19) United States
(12) Patent Application Publication
(10) Pub. No.: US 2026/0245681 A1
(43) Pub. Date: Aug. 20, 2026

(54) MULTIMODAL DATA BASED GENERATION OF QUALITY ASSURED KNOWLEDGE GRAPH AND CONTEXTS FOR USER QUERIES

(71) Applicant: Tata Consultancy Services Limited, Mumbai (IN)

(72) Inventors: Rajat Subhra PAL, Kolkata (IN); Manjira SINHA, Kolkata (IN); Avik GHOSE, Kolkata (IN); Tirthankar DASGUPTA, Kolkata (IN); Smita Elayath VASUDEVAN, Kochi (IN); Avishek HALDER, Kolkata (IN); Tungadri BOSE, Pune (IN)

(73) Assignee: Tata Consultancy Services Limited, Mumbai (IN)

(21) Appl. No.: 19/247,706

(22) Filed: Jun. 24, 2025

(30) Foreign Application Priority Data
Feb. 14, 2025 (IN) 202521012945

(51) Int. Cl. G16H 10/60; G06N 5/022; G06N 5/045; G16H 50/50

(52) U.S. Cl. CPC G16H 10/60; G06N 5/022; G06N 5/045; G16H 50/50

(57) ABSTRACT

Gastrointestinal (GI) tract cancers represent a significant burden on global health, with their diagnosis often posing challenges due to overlapping symptoms and complex etiologies. Conventional methods are inaccurate in differentiating between various GI tract cancers and thus remain a formidable task for clinicians, often leading to delays in diagnosis and suboptimal management. Present disclosure provides a system and a method that receive multimodal data for generating a seed knowledge graph and patterns identification. Dynamic mapping is then performed using the identified patterns on the seed knowledge graph to obtain an updated seed knowledge graph using a deep learning model. The system then fuses the employs the updated seed knowledge graph with the multimodal data being processed to obtain multimodal patient profile. The system employs large language models (LLMs) to analyze patient data and generate insights and explainability, ensuring physicians understand the rationale behind diagnosis and treatment recommendations.

### PRIORITY CLAIM

This U.S. patent application claims priority under 35 U.S.C. § 119 to: Indian Patent Application No. 202521012945, filed on Feb. 14, 2025. The entire contents of the aforementioned application are incorporated herein by reference.

### TECHNICAL FIELD

The disclosure herein generally relates to data analysis, and, more particularly, to multimodal data based generation of quality assured knowledge graph and contexts for user queries.

### BACKGROUND

Gastrointestinal (GI) tract cancers represent a significant burden on global health, with their diagnosis often posing challenges due to overlapping symptoms and complex etiologies. Differential diagnosis, the process of distinguishing between similar conditions based on clinical manifestations, imaging, and laboratory findings, plays a crucial role in guiding appropriate treatment strategies and improving patient outcomes. However, accurately differentiating between various GI tract cancers remains a formidable task for clinicians, often leading to delays in diagnosis and suboptimal management. In the era of rapid technological advancements, integrating artificial intelligence (AI) into clinical practice has become imperative for improving healthcare outcomes.

### SUMMARY

Embodiments of the present disclosure present technological improvements as solutions to one or more of the above-mentioned technical problems recognized by the inventors in conventional systems.

For example, in one aspect, there is provided a processor implemented method for multimodal data based generation of quality assured knowledge graph and contexts for user queries. The method comprises receiving, via one or more hardware processors, multimodal data pertaining to a disease diagnosis from one or more sources; pre-processing, via the one or more hardware processors, the multimodal data to obtain a pre-processed multimodal data; converting, via the one or more hardware processors, the pre-processed multimodal data into a structured knowledge dataset using one or more structuring techniques; creating, via the one or more hardware processors, a seed knowledge graph from the structured knowledge dataset based on one or more medical terms, one or more entities and an associated relationship between one or more symptoms, one or more tests, and one or more diagnosis, and a linkage comprised therein; identifying, by using a deep learning model via the one or more hardware processors, one or more patterns along with an associated confidence score based on an analysis of data comprised in the seed knowledge graph; dynamically performing, by using the deep learning model via the one or more hardware processors, a knowledge mapping on the seed knowledge graph using one or more patient-specific insights comprised therein based on the one or more patterns, to obtain an updated seed knowledge graph; and generating, via the one or more hardware processors, a multimodal patient profile by fusing the pre-processed multimodal data and the updated seed knowledge graph, wherein the multimodal patient profile is indicative of a fused knowledge representation that encapsulates knowledge of one or more diseases and a detailed patient-specific information.

In an embodiment, the method further comprises evaluating quality of relationship of data in the multimodal patient profile based on an associated node centrality and an associated graph connectivity using at least one of (i) one or more confidence scores derived from an accuracy of the deep learning model, (ii) a data provenance of the multimodal data obtained from the one or more sources, and (iii) a clinical validation, to obtain a quality-assured knowledge graph.

In an embodiment, the method further comprises generating one or more embeddings derived from the quality-assured knowledge graph, wherein the one or more embeddings are indexed into a vector database.

In an embodiment, the method further comprises generating one or more human-understandable cross modality explanations associated with the multimodal patient profile based on one or more contributions of one or more modalities comprised in the multimodal data.

In an embodiment, an interactive system is provided based on the one or more human-understandable cross modality explanations for enabling an interpretation of a decision-making process for the disease diagnosis.

In an embodiment, the method further comprises generating, by using the one or more embeddings indexed into the vector database, one or more contexts specific to one or more user queries and one or more prompts generated, wherein the one or more user queries pertain to at least one disease diagnosis, and one or more symptoms.

In another aspect, there is provided a processor implemented system for multimodal data based generation of quality assured knowledge graph and contexts for user queries. The system comprises: a memory storing instructions; one or more communication interfaces; and one or more hardware processors coupled to the memory via the one or more communication interfaces, wherein the one or more hardware processors are configured by the instructions to receive multimodal data pertaining to a disease diagnosis from one or more sources; pre-process the multimodal data to obtain a pre-processed multimodal data; convert the pre-processed multimodal data into a structured knowledge dataset using one or more structuring techniques; create a seed knowledge graph from the structured knowledge dataset based on one or more medical terms, one or more entities and an associated relationship between one or more symptoms, one or more tests, and one or more diagnosis, and a linkage comprised therein; identify, by using a deep learning model, one or more patterns along with an associated confidence score based on an analysis of data comprised in the seed knowledge graph; dynamically perform, by using the deep learning model, a knowledge mapping on the seed knowledge graph using one or more patient-specific insights comprised therein based on the one or more patterns, to obtain an updated seed knowledge graph; and generate a multimodal patient profile by fusing the pre-processed multimodal data and the updated seed knowledge graph, wherein the multimodal patient profile is indicative of a fused knowledge representation that encapsulates knowledge of one or more diseases and a detailed patient-specific information.

In an embodiment, the one or more hardware processors are further configured by the instructions to evaluate quality of relationship of data in the multimodal patient profile based on an associated node centrality and an associated graph connectivity using at least one of (i) one or more confidence scores derived from an accuracy of the deep learning model, (ii) a data provenance of the multimodal data obtained from the one or more sources, and (iii) a clinical validation, to obtain a quality-assured knowledge graph.

In an embodiment, the one or more hardware processors are further configured by the instructions to generate one or more embeddings derived from the quality-assured knowledge graph, wherein the one or more embeddings are indexed into a vector database.

In an embodiment, the one or more hardware processors are further configured by the instructions to generate one or more human-understandable cross modality explanations associated with the multimodal patient profile based on one or more contributions of one or more modalities comprised in the multimodal data.

In an embodiment, an interactive system is provided based on the one or more human-understandable cross modality explanations for enabling an interpretation of a decision-making process for the disease diagnosis.

In an embodiment, the one or more hardware processors are further configured by the instructions to generate, by using the one or more embeddings indexed into the vector database, one or more contexts specific to one or more user queries and one or more prompts generated, wherein the one or more user queries pertain to at least one disease diagnosis, and one or more symptoms.

In yet another aspect, there are provided one or more non-transitory machine-readable information storage mediums comprising one or more instructions which when executed by one or more hardware processors cause multimodal data based generation of quality assured knowledge graph and contexts for user queries by receiving multimodal data pertaining to a disease diagnosis from one or more sources; pre-processing the multimodal data to obtain a pre-processed multimodal data; converting the pre-processed multimodal data into a structured knowledge dataset using one or more structuring techniques; creating a seed knowledge graph from the structured knowledge dataset based on one or more medical terms, one or more entities and an associated relationship between one or more symptoms, one or more tests, and one or more diagnosis, and a linkage comprised therein; identifying, by using a deep learning model, one or more patterns along with an associated confidence score based on an analysis of data comprised in the seed knowledge graph; dynamically performing, by using the deep learning model, a knowledge mapping on the seed knowledge graph using one or more patient-specific insights comprised therein based on the one or more patterns, to obtain an updated seed knowledge graph; and generating a multimodal patient profile by fusing the pre-processed multimodal data and the updated seed knowledge graph, wherein the multimodal patient profile is indicative of a fused knowledge representation that encapsulates knowledge of one or more diseases and a detailed patient-specific information.

In an embodiment, the one or more instructions which when executed by the one or more hardware processors further cause evaluating quality of relationship of data in the multimodal patient profile based on an associated node centrality and an associated graph connectivity using at least one of (i) one or more confidence scores derived from an accuracy of the deep learning model, (ii) a data provenance of the multimodal data obtained from the one or more sources, and (iii) a clinical validation, to obtain a quality-assured knowledge graph.

In an embodiment, the one or more instructions which when executed by the one or more hardware processors further cause generating one or more embeddings derived from the quality-assured knowledge graph, wherein the one or more embeddings are indexed into a vector database.

In an embodiment, the one or more instructions which when executed by the one or more hardware processors further cause generating one or more human-understandable cross modality explanations associated with the multimodal patient profile based on one or more contributions of one or more modalities comprised in the multimodal data.

In an embodiment, an interactive system is provided based on the one or more human-understandable cross modality explanations for enabling an interpretation of a decision-making process for the disease diagnosis.

In an embodiment, the one or more instructions which when executed by the one or more hardware processors further cause generating, by using the one or more embeddings indexed into the vector database, one or more contexts specific to one or more user queries and one or more prompts generated, wherein the one or more user queries pertain to at least one disease diagnosis, and one or more symptoms.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this disclosure, illustrate exemplary embodiments and, together with the description, serve to explain the disclosed principles:

FIG. 1 depicts an exemplary system for multimodal data based generation of quality assured knowledge graph and contexts for user queries, in accordance with an embodiment of the present disclosure.

FIG. 2 depicts an exemplary high level block diagram of the system 100 of FIG. 1 for multimodal data based generation of quality assured knowledge graph and contexts for user queries, in accordance with an embodiment of the present disclosure.

FIG. 3 depicts an exemplary flow chart illustrating a method for multimodal data based generation of quality assured knowledge graph and contexts for user queries, using the systems of FIG. 1-2, in accordance with an embodiment of the present disclosure.

### DETAILED DESCRIPTION

Exemplary embodiments are described with reference to the accompanying drawings. In the figures, the left-most digit(s) of a reference number identifies the figure in which the reference number first appears. Wherever convenient, the same reference numbers are used throughout the drawings to refer to the same or like parts. While examples and features of disclosed principles are described herein, modifications, adaptations, and other implementations are possible without departing from the scope of the disclosed embodiments.

Gastrointestinal (GI) tract cancers represent a significant burden on global health, with their diagnosis often posing challenges due to overlapping symptoms and complex etiologies. Differential diagnosis, the process of distinguishing between similar conditions based on clinical manifestations, imaging, and laboratory findings, plays a crucial role in guiding appropriate treatment strategies and improving patient outcomes. However, accurately differentiating between various GI tract cancers remains a formidable task for clinicians, often leading to delays in diagnosis and suboptimal management. In the era of rapid technological advancements, integrating artificial intelligence (AI) into clinical practice has become imperative for improving healthcare outcomes. Present disclosure provides systems and methods that combine a multimodal knowledge graph with a Large Language Model (LLM)-based clinical assistant to augment physicians'diagnostic and treatment capabilities for differential diagnosis of GI tract Cancer. Convention systems have limitations such as subjective interpretation of symptoms and diagnostic tests, high volume of patient but less time to diagnosis variability in physician expertise, lack of end-to-end traceability, constantly evolving knowledge system quality of citations not assured, pathophysiological basis mining is time consuming. While LLMs are powerful for processing text, their effectiveness relies on the quality and specificity of the knowledge they are trained on. Currently, there is a lack of comprehensive, fine-grained knowledge graphs specifically tailored for GI cancers. Existing knowledge graphs may be insufficient to accurately represent the intricate details of GI cancer subtypes, symptoms, risk factors, and treatment options, hindering the LLM's ability to make precise diagnoses.

Further, reasoning algorithms used within knowledge graphs need to be rigorously evaluated for their accuracy and generalizability within the context of GI cancers. Without proper evaluation, there is a risk that algorithms may make inaccurate inferences or struggle to generalize across diverse patient populations, potentially leading to misdiagnosis. Transparency and interpretability are crucial for building trust in AI-powered medical diagnoses. Currently, there is a lack of effective explainable AI (XAI) techniques specifically tailored for LLM-based GI cancer diagnosis systems. Clinicians need to understand the rationale behind AI recommendations to confidently integrate them into their decision-making. Without XAI, clinicians may be hesitant to rely on AI diagnoses, hindering their adoption.

Further, current approaches primarily focus on textual data. Integrating multimodal knowledge graphs (combining text, images, clinical data) with LLMs is crucial to leveraging a wider range of information for more comprehensive diagnoses. Multimodal integration can improve accuracy by analyzing images from endoscopy, Computed Tomography (CT) scans, and other sources, enabling more accurate diagnoses and personalized treatment strategies.

Systems and methods of the present disclosure address the technology gaps identified above by combining a comprehensive knowledge graph (KG) with large language models (LLMs) and advanced natural language processing (NLP) techniques. Firstly, the system develops Domain-Specific Knowledge Graphs. In other words, a seed knowledge graph is built containing a wide range of medical information specific to GI cancers. This includes symptoms: a taxonomy of symptoms, ranging from common to subtle, associated with various GI cancers; risk factors: Information about factors contributing to GI cancer development, including age, family history, diet, environmental exposures, genetic predispositions, and comorbidities; Diagnostic Tests: Detailed descriptions of imaging studies (CT, MRI, endoscopy), biopsy techniques, tumor markers, and molecular profiling assays used in diagnosing GI cancers; Pathological Findings: Histopathological characteristics specific to different types and stages of GI cancers, covering features like tumor morphology, grade, stage, and molecular biomarkers; Treatment Options: A comprehensive catalog of surgical interventions, chemotherapy regimens, radiation therapy protocols, targeted therapies, immunotherapies, and palliative care strategies specific to each GI cancer type.

Secondly, the generalizability and accuracy of reasoning algorithms is evaluated. The LLM is trained on the knowledge graph (KG), enabling it to perform reasoning tasks specifically tailored to GI cancers. This ensures the LLM's knowledge and reasoning abilities are grounded in relevant medical information. The system's accuracy and generalizability are continuously evaluated and improved through active learning techniques, where the LLM learns from new data and user interactions.

Thirdly, implementing effective Explainable AI (XAI) techniques. The system incorporates XAI mechanisms, providing transparency and trust in the clinical setting. This is achieved through transparency: clear explanations of diagnoses and recommendations are provided, helping physicians understand the AI's reasoning; feature importance: The system highlights key factors contributing to a diagnosis, enabling physicians to validate the AI's insights; confidence scores: Confidence scores are provided for each diagnostic possibility, allowing physicians to assess the certainty of the AI's suggestions; auditable process: A clear audit trail of the reasoning and decision-making process is maintained, allowing for analysis and verification.

Integrating the Multimodal Knowledge Graphs and LLMs: The system of the present disclosure integrates multimodal data into the KG and LLM: The LLM excels in understanding medical language, enabling it to analyze patient records, including textual reports, imaging results, and pathology findings. Information from diverse sources like radiological images, histological slides, and laboratory reports are incorporated into the LLM, thus creating a comprehensive understanding of the patient's clinical status. Sophisticated pattern recognition capabilities of the LLM identify subtle correlations and patterns in medical data, facilitating the detection of diagnostic clues. The LLM infers implicit information and relationships embedded within medical texts, contextualizing clinical findings within the broader patient context. The system integrates microbiome analysis into its approach, analyzing stool samples to evaluate gut microbiota composition as an indicator of cancerous growth. This adds another dimension to the diagnostic process, particularly for early detection.

The system implements active learning techniques that enable the LLM and KG to continuously learn from new data and user interactions, improving performance over time. A federated learning approach allows the MKG and models to be trained and updated collaboratively across multiple healthcare institutions, while preserving patient data privacy. Measures are in place to address bias and toxicity in the LLM and AI models, ensuring fairness and responsible use. Industry-leading security measures are implemented to protect patient data, maintain privacy, and prevent misuse of the system.

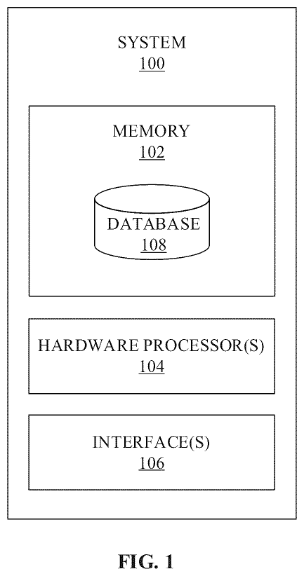

Referring now to the drawings, and more particularly to FIGS. 1 through 3, where similar reference characters denote corresponding features consistently throughout the figures, there are shown preferred embodiments, and these embodiments are described in the context of the following exemplary system and/or method.

FIG. 1 depicts an exemplary system 100 for multimodal data based generation of quality assured knowledge graph and contexts for user queries, in accordance with an embodiment of the present disclosure. In an embodiment, the system 100 includes one or more hardware processors 104, communication interface device(s) or input/output (I/O) interface(s) 106 (also referred as interface(s)), and one or more data storage devices or memory 102 operatively coupled to the one or more hardware processors 104. The one or more processors 104 may be one or more software processing components and/or hardware processors. In an embodiment, the hardware processors can be implemented as one or more microprocessors, microcomputers, microcontrollers, digital signal processors, central processing units, state machines, logic circuitries, and/or any devices that manipulate signals based on operational instructions. Among other capabilities, the processor(s) is/are configured to fetch and execute computer-readable instructions stored in the memory. In an embodiment, the system 100 can be implemented in a variety of computing systems, such as laptop computers, notebooks, hand-held devices (e.g., smartphones, tablet phones, mobile communication devices, and the like), workstations, mainframe computers, servers, a network cloud, and the like.

The I/O interface device(s) 106 can include a variety of software and hardware interfaces, for example, a web interface, a graphical user interface, and the like and can facilitate multiple communications within a wide variety of networks N/W and protocol types, including wired networks, for example, LAN, cable, etc., and wireless networks, such as WLAN, cellular, or satellite. In an embodiment, the I/O interface device(s) can include one or more ports for connecting a number of devices to one another or to another server.

The memory 102 may include any computer-readable medium known in the art including, for example, volatile memory, such as static random-access memory (SRAM) and dynamic-random access memory (DRAM), and/or non-volatile memory, such as read only memory (ROM), erasable programmable ROM, flash memories, hard disks, optical disks, and magnetic tapes. In an embodiment, a database 108 is comprised in the memory 102, wherein the database 108 comprises multimodal data pertaining to a disease diagnosis from one or more sources. The database 108 further comprises structured knowledge dataset derived from the preprocessing of multimodal data, seed knowledge graphs, patterns identified in the seed knowledge graph, multimodal patient profiles, quality of relationship of data in the multimodal patient profile, embeddings derived from the quality-assured knowledge graph, one or more human-understandable cross modality explanations associated with the multimodal patient profile, and the like. The memory 102 further comprises (or may further comprise) information pertaining to input(s)/output(s) of each step performed by the systems and methods of the present disclosure. In other words, input(s) fed at each step and output(s) generated at each step are comprised in the memory 102 and can be utilized in further processing and analysis.

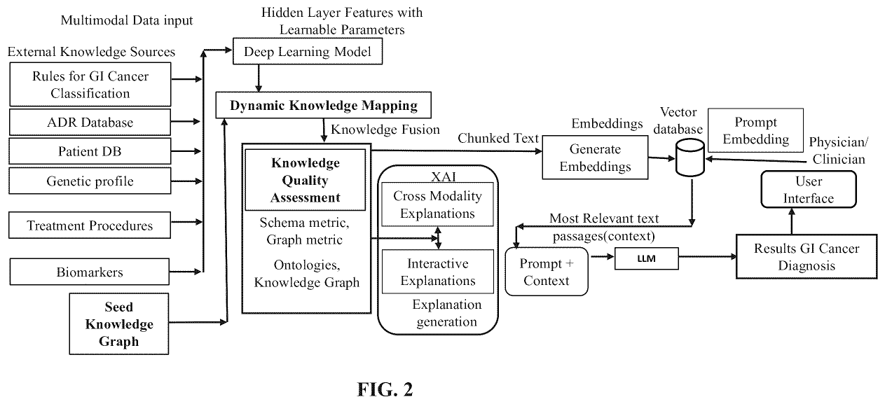

FIG. 2, with reference to FIG. 1, depicts an exemplary high level block diagram of the system 100 of FIG. 1 for multimodal data based generation of quality assured knowledge graph and contexts for user queries, in accordance with an embodiment of the present disclosure.

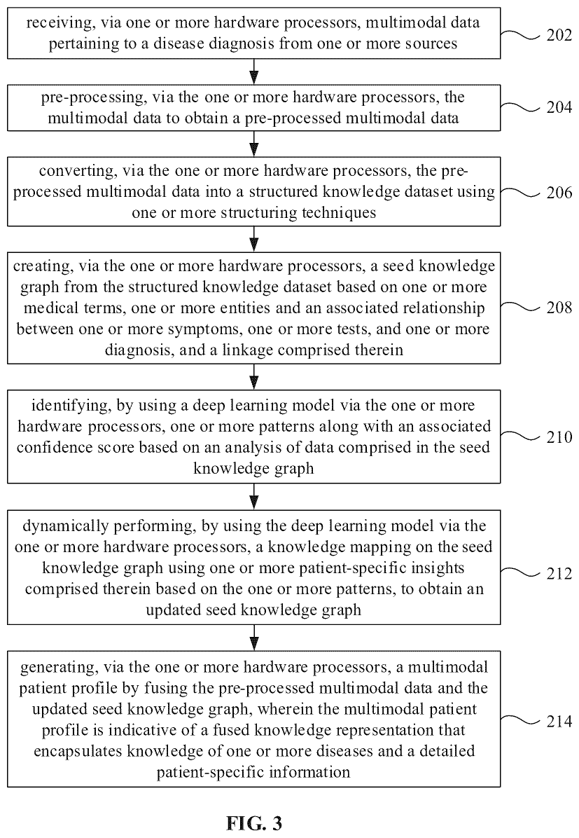

FIG. 3, with reference to FIGS. 1-2, depicts an exemplary flow chart illustrating a method for multimodal data based generation of quality assured knowledge graph and contexts for user queries, using the systems 100 of FIG. 1-2, in accordance with an embodiment of the present disclosure. In an embodiment, the system(s) 100 comprises one or more data storage devices or the memory 102 operatively coupled to the one or more hardware processors 104 and is configured to store instructions for execution of steps of the method by the one or more processors 104. The steps of the method of the present disclosure will now be explained with reference to components of the system 100 of FIG. 1, the block diagram of the system 100 depicted in FIG. 2, and the flow diagram as depicted in FIG. 3. Although process steps, method steps, techniques or the like may be described in a sequential order, such processes, methods, and techniques may be configured to work in alternate orders. In other words, any sequence or order of steps that may be described does not necessarily indicate a requirement that the steps be performed in that order. The steps of processes described herein may be performed in any order practical. Further, some steps may be performed simultaneously.

At step 202 of the method of the present disclosure, the one or more hardware processors 104 receive multimodal data pertaining to a disease diagnosis from one or more sources. For instance, the multimodal data includes, but is not limited to, unstructured clinical text, audio, images, video, genomic data, structured tabular data, lifestyle data, and external knowledge sources. The one or more sources may include various clinical systems such as but are not limited to, electronic health records (EHRs), picture archiving and communication system (PACS), pathology systems, and the like. The unstructured clinical text may include clinical notes, patient records, medical articles, pathology reports, patient history, symptoms, physical exam reports, nurse's notes, and the like. The audio may include patient interviews, physician dictations, medical episodes, and the like. The images may include endoscopic images, CT scans, magnetic resonance imaging (MRI) scans related to the gastrointestinal tract, X-rays, and the like. The video may include recordings of endoscopic procedures, ultrasound, and the like. The genomic data may include mutational profiles, gene expression data, biomarkers specific to GI cancers, and the like. The structured tabular data may include laboratory results such as blood tests, biopsy reports, tumor markers (e.g., CEA, CA 19-9), and the like. The lifestyle data may include smoking, alcohol intake, dietary habits, etc. The external knowledge sources may include research publications, medical databases, clinical guidelines, drug information, and the like. The disease diagnosis in the present disclosure may be referred to as patient with suspected colorectal cancer. It is to be understood by a person having ordinary skill in the art or person skilled in the art that the above examples of multimodal data and the disease described herein shall not be construed as limiting the scope of the present disclosure.

At step 204 of the method of the present disclosure, the one or more hardware processors 104 pre-process the multimodal data to obtain a pre-processed multimodal data. At step 206 of the method of the present disclosure, the one or more hardware processors 104 convert the pre-processed multimodal data into a structured knowledge dataset using one or more structuring techniques. The above steps 204 and 206 are better understood by way of following description:

The system 100 employs one or more natural language processing (NLP) techniques known in the art for processing the text received as an input. This technique includes tokenization, annotation, and structuring of text data such as clinical notes, patient records, and medical articles. The system 100 further employs various image processing models for visual data serving as an input in the multimodal data. This technique includes standardizing formats and enhancing relevant details in images and videos, such as detecting abnormal structures in CT scans. The system 100 further implements speech-to-text models for audio, wherein the audio data is converted into text and relevant clinical information, including emotional markers are extracted. The system 100 further employs various statistical methods known in the art for lab results and genomic data to ensure ensuring uniformity and accordingly generate numerical data for analysis.

The method considered a patient with suspected colon cancer as described above. The received multimodal data may include text such as Doctor's notes: “Patient reports intermittent abdominal pain for the past 3 months, increased frequency of bowel movements, and noticeable fatigue. Family history of colon cancer.”, image: Colonoscopy image showing a suspicious mass, and lab results: Blood test results indicating slightly elevated carcinoembryonic antigen (CEA) levels. Considering the above multimodal data, the pre-processing steps include processing the doctor's notes are processed using NLP, wherein the tokenization is performed by breaking/splitting the text into individual words (e.g., “Patient,” “reports,” “intermittent,” etc.), applying named entity recognition (NER) technique for identifying medical entities like “abdominal pain,” “bowel movements,” and “colon cancer.”, extracting relationship by determining the relationships between entities (e.g., the patient “experiences” abdominal pain), normalizing by converting terms to a standard format (e.g., “abd pain” to “abdominal pain”). Similarly, the colonoscopy image undergoes processing to resize and standardize format thus ensuring compatibility with machine learning model implemented herein. This further includes image enhancements to improve contrast and sharpness to highlight potential abnormalities, applying noise reduction techniques to removing artifacts that could interfere with analysis. The lab results which include CEA levels are normalized by converting to a standard unit of measurement. Further the pre-processing includes comparison of the data with normal reference ranges for conceptualization. Thus, the pre-processed multimodal data includes information that is a structured format suitable for analysis by the deep learning model. This includes structured text data: a representation of the doctor's notes including identified medical entities and their relationships, processed image data: an enhanced and standardized version of the colonoscopy image, and normalized lab data: the CEA level expressed in a standard unit and flagged as potentially elevated. The step of 206 enables the following:
1. Seed Knowledge Graph: This establishes the initial structured knowledge base specifically for GI cancers. Structuring techniques here include named entity recognition (NER), relationship extraction, and attribute extraction from structured, semi-structured, and unstructured data sources (ontologies, medical concepts, research publications).
2. Dynamic Knowledge Mapping: This is where the dynamic aspect of structuring comes into play. The initial Seed Knowledge Graph is updated based on the analysis of the patient's multimodal data. This involves identifying new relationships between entities (e.g., a newly discovered biomarker's connection to a cancer subtype) and updating the strength of existing relationships based on real-world data.
3. Knowledge Fusion: While this step focuses primarily on combining insights from the Dynamic Knowledge Graph and the deep learning analysis, a critical component is the alignment and merging of patient-specific data with the broader medical knowledge.

For instance, the system 100 receives the following pre-processed multimodal data about a patient:
1. Text: “Patient presents with abdominal pain and weight loss. Family history of colon cancer.”
2. Image: Colonoscopy image showing a suspicious polyp.
3. Biomarkers: Elevated CEA levels.

The system 100 starts with its existing knowledge graph containing information about GI cancers, risk factors, symptoms, and diagnostic procedures. Based on the patient data, the system 100 identifies “abdominal pain” and “weight loss” as symptoms related to potential GI cancers within the graph. The “family history of colon cancer” strengthens the connection between the patient and the “colon cancer” node. The image analysis result (suspicious polyp) further increases the probability of a connection to “colon cancer” or related polyp types. The elevated CEA levels are linked as potential biomarkers for colon cancer. The system might dynamically create new relationships or update existing ones in the graph based on current research or emerging trends related to CEA and specific GI cancers.

The system 100 then integrates these individual pieces of information from the updated knowledge graph with the patient's specific data. This creates a unified, structured dataset within the graph, representing a holistic view of the patient's potential colon cancer diagnosis based on all available information.

This final structured knowledge dataset within the dynamic knowledge graph becomes the foundation for subsequent steps like quality assessment and XAI explanations. It ensures that the LLM receives contextually rich and structured information for accurate diagnosis and treatment recommendations. The method and the system 100 of the present disclosure described herein leverage various types of Multimodal LLM Models. The landscape of Multimodal Large Language Models (MLLMs) is also rapidly expanding, offering a diverse range of both open-source and proprietary options. Open-source MLLMs, exemplified by projects like LLaMA, Mistral, and Gemma, provide flexibility and community-driven development. Notable proprietary MLLMs include Google's Gemini, OpenAI's GPT-4 (with its multimodal variant), and GPT-3.5, as well as other significant models like Kosmos-1 and BLIP-2. While proprietary models often offer advanced capabilities and dedicated support, they typically come with licensing fees and usage costs. In contrast, open-source models offer greater accessibility and customizability but may require more technical expertise for implementation and fine-tuning. We can choose between open-source and proprietary MLLMs necessitate careful consideration of factors such as cost, licensing restrictions, performance requirements, and available development resources. This decision is crucial for ensuring the selected model aligns with the specific needs and constraints of our application. It is to be understood by a person having ordinary skill in the art or person skilled in the art that the above examples of LLMs and variants of the same shall not be construed as limiting the scope of the present disclosure.

At step 208 of the method of the present disclosure, the one or more hardware processors 104 create a seed knowledge graph from the structured knowledge dataset based on one or more medical terms, one or more entities and an associated relationship between one or more symptoms, one or more tests, and one or more diagnosis, and a linkage comprised therein. The above step 208 is better understood by way following description:

The system 100 describes the seed knowledge graph as the foundation which contains pre-existing medical knowledge specific to GI cancers. This includes entities such as cancer types, symptoms, biomarkers, risk factors, and treatment options, along with the relationships between them, and the like. Crucially, it incorporates established medical guidelines for GI cancer diagnosis. Considering the example of multimodal data received in step 202, the seed knowledge graph is generated by way of following steps:
1. Data input: The system 100 starts with a variety of data sources, including:
   Ontologies: Standardized vocabularies defining GI cancer-related concepts (e.g., SNOMED CT, UMLS). Example: An ontology might define “colorectal cancer” as a subtype of “GI cancer” and link it to related concepts like “colonoscopy” and “CEA tumor marker.”
   Medical Literature: research papers, clinical reports related to GI cancers. For example, a research paper might describe the relationship between a specific genetic mutation and the increased risk of developing a particular type of GI cancer.
   Clinical guidelines: Established protocols for diagnosing and treating different GI cancers. For example: guidelines might specify the recommended diagnostic pathway for a patient presenting specific symptoms, such as abdominal pain and rectal bleeding.
2. Information extraction (NER, Relationship Extraction, Attribute Extraction):
   Named Entity Recognition (NER): Identifies key medical terms and entities within the input data. Example: From the text “The patient presented with abdominal pain and a history of polyps,” NER would identify “abdominal pain” and “polyps” as medical entities.
   Relationship Extraction: Determines the connections between identified entities. Example: From the sentence “Smoking increases the risk of colorectal cancer,” the system would extract the relationship “increases risk” between “smoking” and “colorectal cancer.”
   Attribute Extraction: Identifies specific characteristics of entities. Example: For the entity “colorectal cancer,” attributes might include “stage,” “location,” and “genetic markers.”
3. Knowledge graph construction: The extracted information is used to build the seed knowledge graph.
   Nodes: Each medical entity becomes a node in the graph. For example, “Colorectal cancer,” “abdominal pain,” “smoking,” “CEA tumor marker.”
   Edges: Relationships between entities are represented as edges connecting the nodes. For example, an edge labeled “increases risk” would connect “smoking” to “colorectal cancer.”
   Properties: Attributes are associated with nodes as properties. Example: The “colorectal cancer” node might have a property “stage” with a value of “II.”

Below description illustrates how the seed knowledge graph is built/generated:

The system 100 processes a clinical guideline stating: “Patients with a family history of Lynch syndrome and experiencing persistent rectal bleeding should undergo a colonoscopy.”
1. NER identifies “Lynch syndrome,” “rectal bleeding,” and “colonoscopy.”
2. Relationship Extraction determines the relationships:
   “family history of” between “patient” and “Lynch syndrome”
   “experiencing” between “patient” and “rectal bleeding”
   “should undergo” between “patient” and “colonoscopy”
3. The knowledge graph is populated with nodes representing “patient,” “Lynch syndrome,” “rectal bleeding,” and “colonoscopy,” connected by edges representing the extracted relationships.

This process is repeated across multiple data sources, thus creating a comprehensive seed knowledge graph that captures established medical knowledge and diagnostic pathways for GI cancers. This forms the basis for subsequent steps, where patient-specific data is integrated and analyzed.

At step 210 of the method of the present disclosure, the one or more hardware processors 104 identify, by using a deep learning model, one or more patterns along with an associated confidence score based on an analysis of data comprised in the seed knowledge graph. The above step 210 of identifying the one or more patterns is better understood by way following description:

The deep learning model performs the core pattern identification using various deep learning models specialized for different data types (text, image, genomic, and the like.). The deep learning model can be transformer based generative deep neural networks for different modalities such as for example, RoBerta for text, vision transformer for image, nucleotide transformer for genomics data. The output is a likelihood of different GI cancers and associated features. This addresses the “identifying one or more patterns” aspect of the step 210. The deep learning model's output updates the seed knowledge graph, refining relationships between entities and strengthening or weakening existing connections. While not directly part of pattern identification, it contributes to contextualizing and refining the patterns found, which implicitly influences the confidence score. The confidence score computation involves implementation of known in the art techniques by the system 100. Further, the system 100 combines the deep learning outputs with the dynamic knowledge graph or the updated seed knowledge graph. The initial part of knowledge fusion focuses on creating a unified view, which includes associating confidence scores with identified patterns. This describes the “associated confidence score” aspect of the step 210.

The deep learning models process the pre-processed multimodal data (from step 204). For example, a machine learning model such as neural networks analyze colonoscopy images to detect tumors, while NLP models analyze clinical notes to extract symptoms. Each model outputs a probabilistic assessment (e.g., 80% likelihood of colon cancer based on the image).
1. The machine learning models also extract relevant features, such as tumor size, location, genetic mutations, specific symptoms, and the like.
2. Dynamic seed knowledge graph update: The extracted features and likelihoods are used to update the seed knowledge graph (from step 208). For example, if a new biomarker is found to be strongly correlated with a specific cancer type in the deep learning analysis, this relationship is added or strengthened in the graph.
3. Knowledge fusion and confidence scoring: The outputs from different deep learning models are combined, considering the updated knowledge graph. This fusion process produces a more refined assessment. Confidence scores are then calculated. A higher confidence score is assigned when multiple modalities and the knowledge graph converge on a particular diagnosis.

The above 3 points are better understood by way of following description:

A patient presents with abdominal pain, changes in bowel habits, and fatigue.
Image analysis (CNN): A colonoscopy image reveals a suspicious mass, giving an 80% likelihood of colon cancer.
Text analysis (NLP): Clinical notes mentioning family history of colon cancer and past polyps increase the likelihood.
Biomarker analysis: Slightly elevated CEA levels add further evidence.
Seed knowledge graph update: The system 100 finds in the seed knowledge graph that the specific symptoms and the patient's age strengthen the link to colon cancer.
Knowledge fusion and confidence score: Combining these findings with the strengthened seed knowledge graph connections results in a high confidence score (e.g., 95%) for a colon cancer diagnosis. The system explains that the high confidence is driven by the image analysis, supported by the patient's symptoms, family history, and biomarker levels.

At step 212 of the method of the present disclosure, the one or more hardware processors 104 dynamically perform, by using the deep learning model, a knowledge mapping on the seed knowledge graph using one or more patient-specific insights comprised therein based on the one or more patterns, to obtain an updated seed knowledge graph. The above step 212 of identifying the one or more patterns is better understood by way following description and steps:
1. Input: The dynamic knowledge mapping receives two primary inputs:
   Outputs from the deep learning model: This includes the likelihood of different GI cancers (classifications) and extracted features (e.g., identified biomarkers, visual features from images, genetic mutations). These are the “patient-specific insights”, and “patterns” referred to in the claim.
   The seed knowledge graph: This is the initial knowledge graph built with general medical knowledge about GI cancers.
2. Process: The deep learning model's output is used to update the seed knowledge graph. This happens in several ways:
   New relationships: If the deep learning model identifies a new correlation in the patient's data (e.g., a specific genetic mutation correlating with a newly discovered biomarker), this relationship is added to the graph.
   Strengthening of existing Relationships: Existing relationships in the seed knowledge graph are strengthened or weakened based on the patient data. For instance, if the patient's data confirms a known link between a symptom and a specific GI cancer, the strength of that relationship in the graph is increased. Conversely, if the patient's data contradicts an existing relationship, its strength might be reduced.
   Ontology-based algorithm implementation: The system 100 ensures that the updates to the seed knowledge graph are consistent with medical ontologies and maintain the graph's structural integrity. This prevents the graph from becoming cluttered with spurious or irrelevant information.
3. Output: The output is an updated seed knowledge graph. This updated seed knowledge graph is now tailored to the specific patient by integrating the general medical knowledge with patient-specific insights derived from their multimodal data.

The above 3 points are better understood by way of example:

Considering that the seed knowledge graph contains a relationship between “abdominal pain” and “colon cancer,” but this relationship is not very strong initially. Now, suppose the deep learning model analyzes a patient's CT scan and detects a tumor in the colon. Simultaneously, the patient reports abdominal pain. The dynamic knowledge mapping technique process would then strengthen the link between “abdominal pain” and “colon cancer” in the updated seed knowledge graph because the patient's specific data reinforces this association. Further, if the deep learning model also detects a rare genetic mutation never before associated with colon cancer in the seed knowledge graph, the dynamic knowledge mapping process creates a new link between the mutation and colon cancer. This dynamic updating makes the knowledge graph more informative and relevant for diagnosing future patients with similar presentations. This updated seed knowledge graph is used in the subsequent steps of the diagnostic process, leading to a more precise and personalized diagnosis. In the dynamic knowledge mapping, apart from adding an edge or increasing the connection weight, there may be situations where an edge may be removed, or the connection strength may be decreased if it is found in a new study that an earlier root cause of cancer is not as effective as it was thought to be.

Referring to steps of FIG. 3, at step 214 of the method of the present disclosure, the one or more hardware processors 104 generate a multimodal patient profile (also referred to as fused knowledge representation and interchangeably used herein) by fusing the pre-processed multimodal data and the updated seed knowledge graph. The 214 step of fusion is better understood by the following description:
1. Objective: To combine multimodal data and dynamic knowledge from the updated seed knowledge graph into a single decision-making framework.
2. Input: The dynamic seed knowledge graph (updated seed knowledge graph) and the extracted insights from the multimodal data (pre-processed multimodal data).
3. Process: Multimodal fusion models combine insights from text, images, lab tests, and genomic data. This step aligns and merges the information from patient-specific data with the broader medical knowledge contained in the Dynamic Knowledge Graph. Cross-validation techniques ensure that conflicting information is analyzed in context.
4. Output: A comprehensive, multimodal patient profile enriched by dynamic knowledge, leading to a highly accurate diagnostic assessment.

The system 100 has considered the colon cancer use case.
1. Pre-processed multimodal data includes processed data from doctor's notes (text), colonoscopy image (image), blood test results (biomarkers), and potentially patient history and lifestyle information.
2. Updated seed knowledge graph: The initial seed knowledge graph containing general information about colon cancer, symptoms, risk factors, etc., is updated with patient-specific information. For instance, the link between the patient's symptoms (abdominal pain, bowel changes), family history of colon cancer, and the “colon cancer” node is strengthened due to the high probability from image analysis. The patient's history of polyps further strengthens this connection.

The knowledge fusion step combines the above two sources of data. The system 100 may use a Bayesian network to represent the probability of colon cancer given the various pieces of evidence. The pre-processed colonoscopy image, indicating a suspicious mass, contributes significantly to the probability. The symptoms, family history, and elevated CEA levels each contribute as well. The updated knowledge graph provides a contextual understanding of how these factors relate to colon cancer. The system 100 weighs the evidence from different modalities and arrives at a fused knowledge representation that expresses a high probability of colon cancer for this specific patient. This fused representation encapsulates both general medical knowledge from the knowledge graph and the specific patient data, creating a multimodal patient profile.

The multimodal patient profile is indicative of a fused knowledge representation that encapsulates knowledge of one or more diseases and a detailed patient-specific information. For instance, the system 100 considered a use case scenario of John Doe, the 60-year-old patient with suspected colon cancer.
1. Patient-specific data: John's multimodal data (symptoms, family history, colonoscopy image, and biomarker results) provide detailed patient-specific information.
2. Disease knowledge: The seed knowledge graph is updated dynamically based on John's case, and this contains knowledge of one or more diseases (specifically colon cancer, its symptoms, risk factors, and treatments). The dynamic update incorporates new potential connections or strengthens existing connections, specific to John's case, like linking his history of polyps to colon cancer.
3. Fusion: The knowledge fusion step combines these two elements. It takes the high probability of colon cancer predicted by the deep learning models based on John's colonoscopy image and the elevated CEA levels and integrates this with the updated knowledge graph which already reflects John's symptoms, family history, and polyp history. This creates a fused knowledge representation.
4. Multimodal Patient Profile (fused knowledge representation): The output of this fusion process is a comprehensive, enriched profile specific to John Doe. It is not just a list of his symptoms and test results but a contextualized representation of his condition, heavily suggesting colon cancer based on the combined patient-specific and general medical knowledge. This enriched profile, reflective of both general and specific knowledge, is indicative of the likelihood of colon cancer.

The system 100 then evaluates quality of relationship of data in the multimodal patient profile based on an associated node centrality and an associated graph connectivity using at least one of (i) one or more confidence scores derived from an accuracy of the deep learning model, (ii) a data provenance of the multimodal data obtained from the one or more sources, and (iii) a clinical validation, to obtain a quality-assured knowledge graph. The step of quality evaluation is better understood by way following description:
1. Multimodal patient profile: This refers to the fused knowledge representation that is created. It combines patient-specific data with the dynamically updated knowledge graph.
2. Associated node centrality and graph connectivity: Refers to “data quality based on metrics like node centrality (how essential an entity is in the graph) and graph connectivity (how well information is linked).”
3. Confidence scores: These are derived from multiple sources:
   Accuracy of the deep learning model: This refers to the confidence scores calculated by the deep learning models known in the art.
   Data provenance: The system 100 performs “provenance tracking” using various technologies including blockchain technology as known in the art, which ensures the reliability and traceability of the data sources.
   Clinical validation: The document describes “clinical feedback loops” where oncologists provide input and validate findings, contributing to the quality assessment.
4. Quality-assured knowledge graph: This is the output of the knowledge quality assessment, representing a refined and validated knowledge graph based on the combined metrics and evaluations.

The system 100 considered that the multimodal patient profile suggests a diagnosis of colon cancer based on the following:
Image analysis (high confidence): A deep learning model identifies a suspicious mass in a colonoscopy image with 90% confidence.
Biomarkers (moderate Confidence): Elevated Cea Levels Are Detected, but these can also be associated with other conditions (60% confidence).
Symptoms (low confidence): The patient reports abdominal pain, which is a non-specific symptom (40% confidence).
Family History (high confidence): The patient has a strong family history of colon cancer (95% confidence).

Based on the above, the system 100 performs the following:
1. Node centrality: The “colon cancer” node in the seed/updated seed knowledge graph has high centrality due to its strong connections with family history, the image finding, and the biomarker. This increases the overall confidence in the diagnosis.
2. Graph connectivity: The system 100 assesses how well the different pieces of evidence are connected. The strong connection between the image finding and family history further strengthens the hypothesis of colon cancer. The weaker connection to the non-specific symptom of abdominal pain is acknowledged.
3. Confidence score aggregation: The system 100 combines the confidence scores from the individual data sources (image, biomarker, symptom) and factors in the data provenance (verified sources for family history and lab results) and potential clinical validation from an oncologist reviewing the case.
4. Quality assurance: Based on these combined factors, the system 100 assigns an overall confidence score to the “colon cancer” diagnosis. Considering that the system 100 has arrived at an 85% confidence score. The quality-assured knowledge graph now contains the “colon cancer” diagnosis linked to the patient's profile with this associated confidence score. This refined graph can then be used for further analysis, explanation generation, and treatment planning.

The system 100 further generates one or more embeddings derived from the quality-assured knowledge graph. The one or more embeddings are indexed into a vector database which is stored in the database 108/memory 102. The above step of generating the one or more embeddings is better understood by way of following description:

Input: Chunked text derived from the quality-assessed knowledge graph related to the patient's case and the initial diagnosis. Output: Numerical vector representations (embeddings) of the chunked text.”
Vector database: “Input: Embeddings generated. Output: Stored embeddings in the Vector Database, indexed for efficient retrieval.”
1. Chunked text: After the updated seed knowledge graph is quality-assured, relevant information pertaining to the patient's specific case and the potential diagnoses is extracted and broken down/split into smaller chunks of text. For instance, if the patient presents abdominal pain and a family history of colon cancer, relevant chunks may include: “abdominal pain,” “family history of colon cancer,” “colonoscopy findings,” “biomarker levels,” descriptions of specific image findings, and the like.
2. Generating embeddings: Each text chunk is converted into a numerical vector representation (an embedding) using one or more embedding models known in the art. The one or more embedding models capture the semantic meaning of the text, so similar concepts have similar vector representations. For example, the embeddings for “colon cancer” and “colorectal carcinoma” would be closer together in vector space than the embedding for “headache.” The system 100 uses “domain-specific, contextualized, and multimodal embeddings.” This means the embeddings are trained or fine-tuned on medical data, potentially even GI-specific data, for better performance. Contextualized embeddings also consider the surrounding words in a sentence for a more nuanced representation. Multimodal embeddings might incorporate information from other data sources, like image features.
3. Indexing into vector database: These embeddings are then stored and indexed within a vector database. This allows for efficient similarity search. When a new query comes in (in the form of a prompt), it is also converted into an embedding. The vector database can then quickly find the most similar embeddings (and corresponding text chunks) from the knowledge graph, providing relevant context to the LLM.

For instance, considering that the patient's colonoscopy image reveals a suspicious polyp. This finding is added to the updated seed knowledge graph and the text chunk “suspicious polyp detected in colonoscopy” is generated. An embedding model creates a vector representation of this chunk. This embedding is stored in the vector database. Later, the LLM prompt may be “What is the most likely diagnosis given the patient's presentation?” This prompt is also converted into an embedding. The vector database quickly identifies the “suspicious polyp” embedding as highly similar and provides the corresponding information to the LLM, influencing its diagnostic reasoning. This demonstrates how embeddings and the vector database bridge the knowledge graph and the LLM, providing crucial context for accurate and informed diagnosis.

The system 100 further generates one or more human-understandable cross modality explanations associated with the multimodal patient profile based on one or more contributions of one or more modalities comprised in the multimodal data. The system 100 uses XAI techniques known in the art to create explanations that detail how each data modality (text, images, audio, etc.) contributed to the final disease diagnosis. The influence of each modality (e.g., “the endoscopic image” shows abnormal growth. Genetic mutation X further increases this risk.”) depicts the associated contributions.

The above step of generating the one or more human-understandable cross modality explanations is better understood by way of following description:

Continuing with the provided colon cancer example:
1. Multimodal Data Input: The system 100 has collected patient data including doctor's notes (text), colonoscopy images, blood test results (biomarkers), and family history.
2. Deep learning model analysis:
   A machine learning technique such as neural network analyzes the colonoscopy image, detecting a suspicious mass with 80% confidence.
   NLP processes the doctor's notes, identifying symptoms like abdominal pain and changes in bowel habits.
   Biomarker analysis notes the slightly elevated CEA levels.
3. Knowledge mapping/fusion: The system 100 integrates these findings with the knowledge graph, which contains information about colon cancer risk factors, symptoms, and diagnostic procedures.
4. XAI-Cross Modality Explanations: Now, the system 100 generates the following explanation for the physician:
   “The high probability of colon cancer is primarily driven by the colonoscopy image analysis (80% confidence), which identified a suspicious mass. This finding is further supported by the patient's reported symptoms of abdominal pain and changes in bowel habits, extracted from the clinical notes. The slightly elevated CEA levels, while not conclusive on their own, contribute to the overall assessment. The patient's family history of colon cancer, as documented in the medical history, also increases the likelihood of this diagnosis.”

This explanation demonstrates how the system 100 generated the cross-modality explanation:
The system 100 identifies each modality (colonoscopy image, clinical notes, biomarkers, family history).
The system 100 quantifies the contribution of the image analysis with a confidence score.
The system 100 explains how each modality supports the diagnosis.

An interactive system/interface is provided by the system 100 based on the one or more human-understandable cross modality explanations for enabling an interpretation of a decision-making process for the disease diagnosis. Clinicians can query the system, explore different scenarios, and receive tailored explanations, enhancing their understanding of the diagnostic process. The system 100 provides a platform/user interface through which the interactive explanations are delivered. It enables the clinicians to interact with the system 100, input data, and review results. For instance, in the provided user interface/platform, the stakeholders such as the clinicians may perform various actions such as clicks on different aspects (e.g., a genetic mutation or an image finding) to get detailed explanations.

The system 100 considers the Colon Cancer example, focusing on how the interactive system empowers interpretation:
1. Initial Diagnosis: The system 100 suggests a high probability of colon cancer based on the combined analysis of colonoscopy image, patient symptoms, family history, and elevated CEA levels.
2. Interactive exploration: Physicians, using the interactive dashboard/interface, click on the “CEA levels” component of the explanation.
3. Tailored explanation: The system 100 responds with a more detailed explanation: “The patient's CEA level is slightly elevated. While this can be indicative of colon cancer, it is important to note that CEA can also be elevated in other conditions like inflammatory bowel disease (IBD). However, the presence of the mass in the colonoscopy image, combined with the patient's family history and symptoms, significantly increases the likelihood of colon cancer compared to IBD. The system's confidence in the colon cancer diagnosis is 85%, considering all modalities. The system 100 may provide an option such as “Click here” to see a comparison of CEA levels in colon cancer versus Inflammatory bowel disease (IBD).”
4. Further interaction: The physicians can further perform various actions such as clicks on the “colonoscopy image” component. The system 100 displays the image with the identified mass highlighted, along with a description: “The CNN identified an irregular mass with spiculated margins in the sigmoid colon, which is highly suggestive of malignancy.” (not shown in FIGS.) The system 100 may further provide zoomed-in views or comparisons with images of benign polyps.
5. What-If Analysis: The system 100 provides a what-if analysis feature through the interface which enables the physicians to ask: “What if the CEA level was normal?” The system dynamically recalculates and responds: “If the CEA level was normal, the system's confidence in the colon cancer diagnosis would decrease to 70%, primarily based on the image findings and other factors. Further investigation, such as a biopsy, is still strongly recommended.”

Thus, the interactive system allows the physicians to interpret the AI's decision-making process. By exploring different aspects of the diagnosis and seeing how the system 100 responds to changes in input or queries, the physicians gain a deeper understanding of the rationale behind the diagnosis and the relative importance of different modalities.

The system 100 further generates, by using the one or more embeddings indexed into the vector database, one or more contexts specific to one or more user queries and one or more prompts generated. The one or more user queries pertain to at least one disease diagnosis, and one or more symptoms. The above step of contexts generation is better understood by way of following description:
First, the embeddings are generated from the chunked text derived from the quality-assured knowledge graph. These embeddings are the numerical representations used in the vector database.
The generated embeddings are indexed into the vector database. This allows for efficient retrieval of relevant information based on one or more similarity searches.
Prompt+Context: This is where the user query comes into play. The user interface provided by the system 100 receives one or more user queries, which are then used to retrieve relevant context from the vector database based on similarity to prompt embeddings. This retrieved context is combined with the initial prompt to form a more refined and context-rich prompt for the LLM.
User interface: This step facilitates the interaction and provides the context from the vector database based on prompt embeddings.

For instance, consider an example where a physician (user) is reviewing the case of a patient with suspected Crohn's disease, a type of inflammatory bowel disease (IBD). The physician may input a query into the system such as: “What are the key differentiating factors between Crohn's disease and ulcerative colitis (another type of IBD) in terms of endoscopic findings?”.

The above example is processed by the system 100 by way of the following steps:
1. User query to Embeddings: The user interface converts the physician's query into an embedding.
2. Context retrieval from vector database: This query embedding is used to search the vector database. The database retrieves embeddings of text chunks related to Crohn's disease, ulcerative colitis, and endoscopic findings, which are semantically similar to the query embedding.
3. Contextualized prompt generation: The retrieved embeddings are converted back into text, forming the “context.” This context is then combined with a pre-defined prompt template to create a complete prompt for the LLM. For instance, the prompt may be: “Given the following information about Crohn's disease, ulcerative colitis, and endoscopic findings: [INSERT RETRIEVED CONTEXT], what are the key differentiating factors between the two conditions based on endoscopic appearance?”
4. LLM processing: The LLM receives this context-rich prompt and generates a response outlining the key endoscopic differences between Crohn's disease and ulcerative colitis.

In essence, the above steps describe the system's ability to use the vector database to dynamically generate relevant context for a given user query (e.g., the physician can query about specific symptoms, differential diagnoses, or request further clarification on any aspect of the diagnostic process). These queries are then used to generate refined prompts for the LLM, leading to a more informative and targeted response from the LLM. This process enhances the diagnostic accuracy and efficiency of the system by providing the LLM with the most pertinent information for each specific case.

Consider that the patient presents with abdominal pain and changes in bowel habits. The initial LLM output suggests the possibility of irritable bowel syndrome (IBS) or Crohn's disease. User Query: “What evidence supports Crohn's disease over IBS, given the patient's family history of colon cancer?”

Below steps describe how the user query is processed by the system:
1. User interface: Receives the query.
2. Embedding generation: The query is converted into an embedding.
3. Vector database: The embedding is used to search the vector database for relevant information, such as research articles on the association between Crohn's disease and colon cancer, the differentiation of IBS and Crohn's, and perhaps even similar patient cases.
4. Prompt generation: A new prompt is constructed, incorporating the original patient information, the initial diagnoses (IBS and Crohn's), and the newly retrieved context regarding family history of colon cancer and its relevance to Crohn's. This refined prompt is then sent to the LLM.
5. LLM processing output: Processes the refined prompt and provides an updated output, potentially highlighting the increased likelihood of Crohn's given the family history and suggesting further investigations to confirm the diagnosis. It may also offer details on why the family history of colon cancer is more relevant to Crohn's than IBS.
6. User interface: Presents the LLM's refined output to the user.
   The system 100 and method of the present disclosure address the technology gaps by integrating a domain-specific KG with advanced NLP techniques, utilizing LLMs for reasoning and analysis, and incorporating XAI mechanisms for transparency and trust. This approach offers a powerful tool for enhancing diagnostic reasoning, improving accuracy, and ultimately aiding in the early detection and treatment of GI cancers. The method and system also enable collecting stool samples from patients/individuals undergoing screening for evaluating their gut microbiota composition, wherein the gut microbiota composition is indicative of the site/type of cancerous growth. For example, while an overabundance of Fusobacteria, Bacteroidetes, Patescibacteria in the gut microbiota may be indicative of signet-ring cell carcinoma (SRCC), higher proportion of Proteobacteria and Acidobacteria may indicate adenocarcinoma (ADC). The method for obtaining the gut microbiota composition of a patient/an individual undergoing screening includes collection of stool samples, extraction of microbial DNA from the collected stool sample, sequencing of the extracted microbial genetic material (DNA) using one of the available DNA sequencing platforms (e.g., Illumina, Oxford Nanopore, etc.) and applying an appropriate bioinformatic pipeline to analyze the sequenced microbial DNA data. The sequencing may be performed using either an amplicon-based approach or a shotgun metagenomic approach. The method further comprises of creating a baseline microbiome composition dataset from healthy individuals. It may be used for comparison/discriminatory analysis to identify the overabundant/ underrepresented microbial taxonomy in the analyzed sample from a patient/an individual undergoing screening when compared to baseline microbiome composition. Thus, the system and method 100 provide a multimodal patient profile specific to a disease diagnosis (e.g., GI cancers) with (i) special emphasize that the deep learning models, knowledge graph, and feature extraction are specifically trained and tailored for GI cancers, (ii) dynamic generation of seed knowledge graph which continuously evolves and refines itself based on new data, improving diagnostic accuracy over time, and (iii) explainable confidence scores that allow stakeholders such as clinicians to understand the reasoning behind the diagnosis.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

It is to be understood that the scope of the protection is extended to such a program and in addition to a computer-readable means having a message therein; such computer-readable storage means contain program-code means for implementation of one or more steps of the method, when the program runs on a server or mobile device or any suitable programmable device. The hardware device can be any kind of device which can be programmed including e.g., any kind of computer like a server or a personal computer, or the like, or any combination thereof. The device may also include means which could be e.g., hardware means like e.g., an application-specific integrated circuit (ASIC), a field-programmable gate array (FPGA), or a combination of hardware and software means, e.g., an ASIC and an FPGA, or at least one microprocessor and at least one memory with software processing components located therein. Thus, the means can include both hardware means and software means. The method embodiments described herein could be implemented in hardware and software. The device may also include software means. Alternatively, the embodiments may be implemented on different hardware devices, e.g., using a plurality of CPUs.

The embodiments herein can comprise hardware and software elements. The embodiments that are implemented in software include but are not limited to, firmware, resident software, microcode, etc. The functions performed by various components described herein may be implemented in other components or combinations of other components. For the purposes of this description, a computer-usable or computer readable medium can be any apparatus that can comprise, store, communicate, propagate, or transport the program for use by or in connection with the instruction execution system, apparatus, or device.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope of the disclosed embodiments. Also, the words “comprising,” “having,” “containing,” and “including,” and other similar forms are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., be non-transitory. Examples include random access memory (RAM), read-only memory (ROM), volatile memory, nonvolatile memory, hard drives, CD ROMs, DVDs, flash drives, disks, and any other known physical storage media.

It is intended that the disclosure and examples be considered as exemplary only, with a true scope of disclosed embodiments being indicated by the following claims.

What is claimed is:

1. A processor implemented method, comprising:
receiving, via one or more hardware processors, multimodal data pertaining to a disease diagnosis from one or more sources;
pre-processing, via the one or more hardware processors, the multimodal data to obtain a pre-processed multimodal data;
converting, via the one or more hardware processors, the pre-processed multimodal data into a structured knowledge dataset using one or more structuring techniques;
creating, via the one or more hardware processors, a seed knowledge graph from the structured knowledge dataset based on one or more medical terms, one or more entities and an associated relationship between one or more symptoms, one or more tests, and one or more diagnosis, and a linkage comprised therein;
identifying, by using a deep learning model via the one or more hardware processors, one or more patterns along with an associated confidence score based on an analysis of data comprised in the seed knowledge graph;
dynamically performing, by using the deep learning model via the one or more hardware processors, a knowledge mapping on the seed knowledge graph using one or more patient-specific insights comprised therein based on the one or more patterns, to obtain an updated seed knowledge graph; and
generating, via the one or more hardware processors, a multimodal patient profile by fusing the pre-processed multimodal data and the updated seed knowledge graph, wherein the multimodal patient profile is indicative of a fused knowledge representation that encapsulates knowledge of one or more diseases and a detailed patient-specific information.

2. The processor implemented method of claim 1, further comprising evaluating quality of relationship of data in the multimodal patient profile based on an associated node centrality and an associated graph connectivity using at least one of (i) one or more confidence scores derived from an accuracy of the deep learning model, (ii) a data provenance of the multimodal data obtained from the one or more sources, and (iii) a clinical validation, to obtain a quality-assured knowledge graph.

3. The processor implemented method of claim 1, further comprising generating one or more embeddings derived from the quality-assured knowledge graph, wherein the one or more embeddings are indexed into a vector database.

4. The processor implemented method of claim 1, further comprising generating one or more human-understandable cross modality explanations associated with the multimodal patient profile based on one or more contributions of one or more modalities comprised in the multimodal data.

5. The processor implemented method of claim 4, wherein an interactive system is provided based on the one or more human-understandable cross modality explanations for enabling an interpretation of a decision-making process for the disease diagnosis.

6. The processor implemented method of claim 3, further comprising generating, by using the one or more embeddings indexed into the vector database, one or more contexts specific to one or more user queries and one or more prompts generated, wherein the one or more user queries pertain to at least one disease diagnosis, and one or more symptoms.

7. A system, comprising:
a memory storing instructions;
one or more communication interfaces; and
one or more hardware processors coupled to the memory via the one or more communication interfaces, wherein the one or more hardware processors are configured by the instructions to:
receive multimodal data pertaining to a disease diagnosis from one or more sources;
pre-process the multimodal data to obtain a pre-processed multimodal data;
convert the pre-processed multimodal data into a structured knowledge dataset using one or more structuring techniques;
create a seed knowledge graph from the structured knowledge dataset based on one or more medical terms, one or more entities and an associated relationship between one or more symptoms, one or more tests, and one or more diagnosis, and a linkage comprised therein;
identify, by using a deep learning model, one or more patterns along with an associated confidence score based on an analysis of data comprised in the seed knowledge graph;
dynamically perform, by using the deep learning model, a knowledge mapping on the seed knowledge graph using one or more patient-specific insights comprised therein based on the one or more patterns, to obtain an updated seed knowledge graph; and
generate a multimodal patient profile by fusing the pre-processed multimodal data and the updated seed knowledge graph, wherein the multimodal patient profile is indicative of a fused knowledge representation that encapsulates knowledge of one or more diseases and a detailed patient-specific information.

8. The system of claim 7, wherein the one or more hardware processors are further configured by the instructions to evaluate quality of relationship of data in the multimodal patient profile based on an associated node centrality and an associated graph connectivity using at least one of (i) one or more confidence scores derived from an accuracy of the deep learning model, (ii) a data provenance of the multimodal data obtained from the one or more sources, and (iii) a clinical validation, to obtain a quality-assured knowledge graph.

9. The system of claim 7, wherein the one or more hardware processors are further configured by the instructions to generate one or more embeddings derived from the quality-assured knowledge graph, wherein the one or more embeddings are indexed into a vector database.

10. The system of claim 7, wherein the one or more hardware processors are further configured by the instructions to generate one or more human-understandable cross modality explanations associated with the multimodal patient profile based on one or more contributions of one or more modalities comprised in the multimodal data.

11. The system of claim 10, wherein an interactive system is provided based on the one or more human-understandable cross modality explanations for enabling an interpretation of a decision-making process for the disease diagnosis.

12. The system of claim 9, wherein the one or more hardware processors are further configured by the instructions to generate, by using the one or more embeddings indexed into the vector database, one or more contexts. specific to one or more user queries and one or more prompts generated, wherein the one or more user queries pertain to at least one disease diagnosis, and one or more symptoms.

13. One or more non-transitory machine-readable information storage mediums comprising one or more instructions which when executed by one or more hardware processors cause:
receiving multimodal data pertaining to a disease diagnosis from one or more sources;
pre-processing the multimodal data to obtain a pre-processed multimodal data;
converting the pre-processed multimodal data into a structured knowledge dataset using one or more structuring techniques;
creating a seed knowledge graph from the structured knowledge dataset based on one or more medical terms, one or more entities and an associated relationship between one or more symptoms, one or more tests, and one or more diagnosis, and a linkage comprised therein;
identifying, by using a deep learning model, one or more patterns along with an associated confidence score based on an analysis of data comprised in the seed knowledge graph;
dynamically performing, by using the deep learning model, a knowledge mapping on the seed knowledge graph using one or more patient-specific insights comprised therein based on the one or more patterns, to obtain an updated seed knowledge graph; and
generating a multimodal patient profile by fusing the pre-processed multimodal data and the updated seed knowledge graph, wherein the multimodal patient profile is indicative of a fused knowledge representation that encapsulates knowledge of one or more diseases and a detailed patient-specific information.

14. The one or more non-transitory machine-readable information storage mediums of claim 13, wherein the one or more instructions which when executed by the one or more hardware processors further cause evaluating quality of relationship of data in the multimodal patient profile based on an associated node centrality and an associated graph connectivity using at least one of (i) one or more confidence scores derived from an accuracy of the deep learning model, (ii) a data provenance of the multimodal data obtained from the one or more sources, and (iii) a clinical validation, to obtain a quality-assured knowledge graph.

15. The one or more non-transitory machine-readable information storage mediums of claim 13, wherein the one or more instructions which when executed by the one or more hardware processors further cause generating one or more embeddings derived from the quality-assured knowledge graph, wherein the one or more embeddings are indexed into a vector database.

16. The one or more non-transitory machine-readable information storage mediums of claim 13, wherein the one or more instructions which when executed by the one or more hardware processors further cause generating one or more human-understandable cross modality explanations associated with the multimodal patient profile based on one or more contributions of one or more modalities comprised in the multimodal data.

17. The one or more non-transitory machine-readable information storage mediums of claim 16, wherein an interactive system is provided based on the one or more human-understandable cross modality explanations for enabling an interpretation of a decision-making process for the disease diagnosis.

18. The one or more non-transitory machine-readable information storage mediums of claim 15, wherein the one or more instructions which when executed by the one or more hardware processors further cause generating, by using the one or more embeddings indexed into the vector database, one or more contexts specific to one or more user queries and one or more prompts generated, wherein the one or more user queries pertain to at least one disease diagnosis, and one or more symptoms.
